(19) United States
(12) Patent Application Publication
(10) Pub. No.: US 2026/0248855 A1
(43) Pub. Date: Aug. 27, 2026

(54) USE OF TRANSFORMING GROWTH FACTOR BETA 1 (TGF-BETA1)-OVEREXPRESSING OLFACTORY MUCOSA MESENCHYMAL STEM CELL (OM-MSC) IN PREPARATION OF DRUG FOR TREATING PARKINSON'S DISEASE (PD)

(71) Applicants: Ming Lu, Changsha, Hunan (CN); Hunan Bizu Biotechnology Co., Ltd., Changsha, Hunan (CN)

(72) Inventors: Ming Lu, Changsha, Hunan (CN); Yi Zhuo, Changsha, Hunan (CN); Wen Lu, Changsha, Hunan (CN); Xuan Li, Changsha, Hunan (CN)

(21) Appl. No.: 18/865,551

(22) PCT Filed: Mar. 15, 2024
(86) PCT No.: PCT/CN2024/081939

§ 371 Date: Nov. 13, 2024

(30) Foreign Application Priority Data
Mar. 16, 2023 (CN) 202310254357.1

(51) Int. Cl. A61K 35/28; A61P 25/16; C07K 14/495; C12N 5/0775

(52) U.S. Cl. CPC A61K 35/28; C12N 5/0668; A61P 25/16; C07K 14/495; C12N 2510/00

(57) ABSTRACT

Use of a transforming growth factor beta 1 (TGF-β1)-overexpressing olfactory mucosa mesenchymal stem cell (OM-MSC) in preparation of a drug for preventing and/or treating Parkinson's disease (PD) is provided, belonging to the technical field of drug preparation. The TGF-β1-overexpressing OM-MSCs can be used to better conduct a neural repair treatment of the PD. By verifying effects of the TGF-β1-overexpressing OM-MSCs in a PD cell model and a PD animal model, it is proved that the TGF-β1-overexpressing OM-MSCs do have a therapeutic effect on PD.

The present application claims priority to Chinese Patent Application No. 202310254357.1 filed to the China National Intellectual Property Administration (CNIPA) on Mar. 16, 2023 and entitled “USE OF TRANSFORMING GROWTH FACTOR BETA 1 (TGF-β1)-OVEREXPRESSING OLFACTORY MUCOSA MESENCHYMAL STEM CELL (OM-MSC) IN PREPARATION OF DRUG FOR TREATING PARKINSON'S DISEASE (PD)”, which is incorporated herein by reference in its entirety.

### TECHNICAL FIELD

The present disclosure belongs to the technical field of drug preparation, and specifically relates to use of a transforming growth factor beta 1 (TGF-β1)-overexpressing olfactory mucosa mesenchymal stem cell (OM-MSC) in preparation of a drug for treating Parkinson's disease (PD).

### BACKGROUND

Parkinson's disease (PD) is a neurodegenerative disease characterized by the loss of dopaminergic neurons (DNs) in the substantia nigra and striatum. The activation of neuroimmune inflammatory response plays a key role in the onset and progression of PD. Alphα-synuclein (α-synuclein) is the main pathological marker of PD. The aggregation of α-synuclein in the substantia nigra and striatum can activate microglia to cause neuroinflammatory response. Activated microglia can act as antigen-presenting cells and regulate effector T cells to accelerate the mitochondrial dysfunction and degeneration of DNs.

The current treatments for PD still have many limitations. With the deepening of stem cell research, stem cell therapy has become a novel strategy for treating PD in recent years. However, the efficacy of current stem cell therapy products still remains limited.

### SUMMARY

An objective of the present disclosure is to provide use of a TGF-β1-overexpressing OM-MSC in preparation of a drug for treating PD. The TGF-β1-overexpressing OM-MSC can be used for more effective neural repair treatment in PD.

The present disclosure provides use of a TGF-β1-overexpressing OM-MSC in preparation of a drug for treating PD.

The present disclosure further provides use of a TGF-β1-overexpressing OM-MSC in preparation of a drug with a protective effect on a neuron.

The present disclosure further provides use of a TGF-β1-overexpressing OM-MSC in preparation of a drug for improving a DN function.

The present disclosure further provides use of a TGF-β1-overexpressing OM-MSC in preparation of a drug with a capacity to scavenge α-synuclein in microglia.

The present disclosure further provides use of a TGF-β1-overexpressing OM-MSC in preparation of a drug with an immunoregulatory effect.

In some embodiments, the immunoregulatory effect includes promoting transformation of a microglial cell from a pro-inflammatory M1 type into an anti-inflammatory M2 type.

In some embodiments, a method for preparing the TGF-β1-overexpressing OM-MSC includes: subjecting an OM-MSC to transfection with a TGF-β1-overexpressing plasmid.

In some embodiments, the TGF-β1-overexpressing plasmid is constructed based on a pcDNA3.1-3×Flag plasmid.

In some embodiments, the transfection is conducted when the OM-MSC is passaged to a fourth generation.

In some embodiments, the TGF-β1-overexpressing plasmid has a mass concentration of 3 μg/mL to 5 μg/mL and the OM-MSC has a viable count of 1×10⁵ cells/10 μL during the transfection.

The present disclosure provides use of a TGF-β1-overexpressing OM-MSC in preparation of a drug for preventing and/or treating PD. Effects of TGF-β1-overexpressing OM-MSC intervention on a PD model are observed, confirming that the TGF-β1-overexpressing OM-MSC may be used as one of the effective treatment approaches for PD. The TGF-β1-overexpressing OM-MSC may be used for enhanced neural repair treatment ub PD.

### BRIEF DESCRIPTION OF THE DRAWINGS

To describe the technical solutions in the examples of this application or in the prior art more clearly, the following briefly describes the accompanying drawings required for the examples. Apparently, the accompanying drawings in the following description show merely some embodiments of this application, and a person of ordinary skill in the art may still derive other accompanying drawings from these accompanying drawings without creative efforts.

FIG. 1 shows identification results of the OM-MSCs provided by the present disclosure;

FIG. 2 shows a protective effect of the TGF-β1-overexpressing OM-MSCs provided by the present disclosure on DN;

FIG. 3 shows results of the TGF-β1-overexpressing OM-MSCs provided by the present disclosure in clearing α-synuclein in microglia;

FIG. 4 shows an immunoregulatory effect of the TGF-β1-overexpressing OM-MSCs provided by the present disclosure on microglia;

FIG. 5 shows neurological function scores of the PD mouse model treated with TGF-β1-overexpressing OM-MSCs transplantation provided by the present disclosure; and

FIG. 6 shows a protective effect of the TGF-β1-overexpressing OM-MSCs provided by the present disclosure on DNs in the substantia nigra of PD mice.

### DETAILED DESCRIPTION OF THE EMBODIMENTS

The present disclosure provides use of a TGF-β1-overexpressing OM-MSC in preparation of a drug for treating PD.

Compared with other mesoderm-derived MSCs, OM-MSCs have the advantages of stable source, high expansion efficiency, and desirable safety, and are derived from the ectoderm homologous to nervous system. The TGF-β1-overexpressing OM-MSC can be used for enhanced neural repair treatment in PD. By verifying effects of TGF-β1-overexpressing OM-MSCs in a PD cell model and a PD animal model in the present disclosure, it is proved that the TGF-β1-overexpressing OM-MSC does have a therapeutic effect on PD. That is, the protein factor TGF-β1 secreted by the TGF-1-overexpressing OM-MSC is significantly upregulated, and can significantly improve the immunoregulatory effect after cell transplantation, thereby significantly enhancing the ability of OM-MSCs to treat PD.

In the present disclosure, the overexpression modification is performed by transfecting an OM-MSC cultured under conventional conditions with TGF-β1-overexpressing plasmid. A method for preparing the TGF-β1-overexpressing OM-MSC includes preferably: subjecting an OM-MSC to transfection with TGF-β1-overexpressing plasmid. The TGF-β1-overexpressing plasmid is constructed based on a pcDNA3.1-3×Flag plasmid. That is, the reagent for overexpressing TGF-β1 to modify OM-MSCs is pcDNA3.1-TGF-β1-3×Flag expression plasmid. The TGF-β1 is preferably human TGF-β1. The TGF-β1 preferably has the nucleotide sequence set forth in positions 879 to 2,051 of the sequence with an NCBI accession number NM_000660.7. In some embodiments, the transfection is conducted on OM-MSCs after passage. More preferably, the transfection is conducted when the OM-MSC is passaged to a fourth generation. In some embodiments, the TGF-β1-overexpressing plasmid has a mass concentration of preferably 3 μg/mL to 5 μg/mL and the OM-MSCs have a viable count of preferably 1×10⁵ cells/μL during the transfection. The culture scheme of TGF-β1-overexpressing OM-MSC can not only improve the adaptation of OM-MSCs to the harsh local microenvironment after transplantation and increase their survival rate, but also enhance paracrine effect and immunoregulatory effect, thereby enhancing the therapeutic effect on neuroimmune-related diseases such as PD.

In the present disclosure, a method of administration of the drug preferably includes intraspinal injection and intravenous injection.

The present disclosure further provides use of a TGF-β1-overexpressing OM-MSC in preparation of a drug with a protective effect on a neuron. In the present disclosure, the TGF-β1 overexpression modification and the drug administration method are preferably the same as described above. OM-MSCs have a certain protective effect on neurons, and the TGF-β1-overexpressing OM-MSCs shows a significantly-enhanced neuroprotective function.

The present disclosure further provides use of a TGF-β1-overexpressing OM-MSC in preparation of a drug for improving DN function. In the present disclosure, the TGF-β1 overexpression modification and the drug administration method are preferably the same as described above.

The present disclosure further provides use of a TGF-β1-overexpressing OM-MSC in preparation of a drug with a capacity to scavenge α-synuclein in microglia. In the present disclosure, the TGF-β1 overexpression modification and the drug administration method are preferably the same as described above. OM-MSCs have a certain scavenging capacity on α-synuclein in microglia, and the TGF-β1-overexpressing OM-MSC has a significantly-enhanced scavenging capacity.

The present disclosure further provides use of a TGF-β1-overexpressing OM-MSC in preparation of a drug with an immunoregulatory effect. In the present disclosure, the TGF-β1 overexpression modification and the drug administration method are preferably the same as described above. The immunoregulatory effect includes preferably promoting transformation of a microglial cell from a pro-inflammatory M1 type into an anti-inflammatory M2 type. OM-MSCs can exert their immunoregulatory effect to promote the transformation of microglia from the pro-inflammatory M1 type to the anti-inflammatory M2 type, and the TGF-β1-overexpressing OM-MSCs have a more significant immunoregulatory effect.

In order to further illustrate the present disclosure, the use of a TGF-β1-overexpressing OM-MSC in preparation of a drug for treating PD provided by the present disclosure will be described in detail below with reference to the examples, but they should not be construed as limiting the protection scope of the present disclosure.

### Example 1

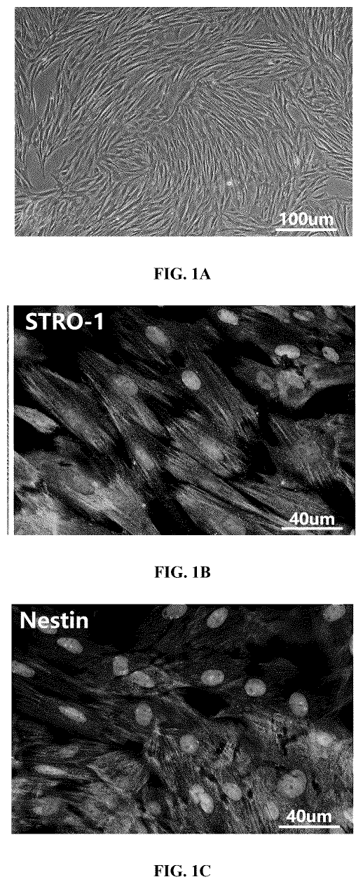

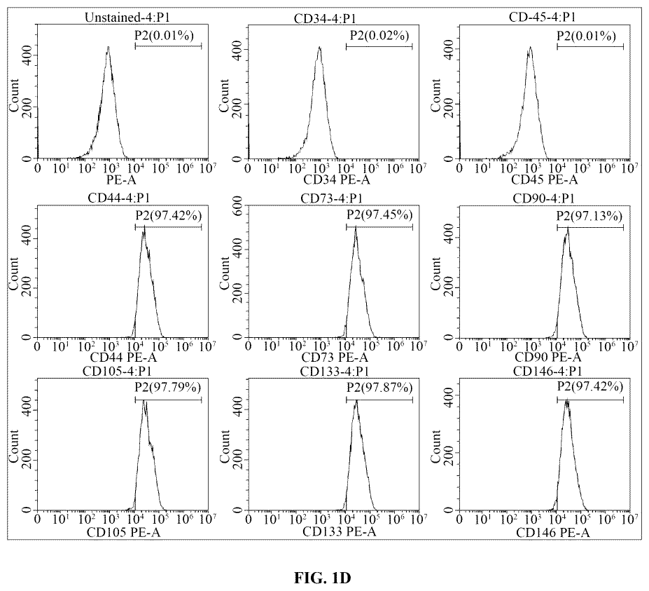

The isolation, culture and identification of OM-MSCs were performed as described in the previous patent: 201410228728.X. OM-MSCs could be used for this experimental study when they were passed to the 4th generation. The results of cell immunofluorescence detection showed that they could express STRO-1 and Nestin at the same time, and the cell surface markers were detected by flow cytometry. The flow cytometry showed that OM-MSCs did not express hematopoietic stem cell markers CD34 and CD45, but expressed adhesion molecules and matrix cell markers CD44, CD73, CD90, CD105, CD133, and CD146, with a purity up to 97% (FIG. 1 shows the identification results of OM-MSCs, where A is the light microscope image of OM-MSCs; B is the fluorescence staining image of MSCs marker STRO-1; C is the fluorescence staining image of neural stem cell marker Nestin; D is the flow cytometry result of MSCs surface markers. The results proved that OM-MSCs highly expressed markers CD44, CD73, CD90, CD105, CD133, and CD146, but did not express markers CD34 and CD45, and had a cell purity over 97%).

Optimized culture scheme of OM-MSCs: when the OM-MSCs were passaged to the 4th generation, they were modified using a TGF-β1-overexpressing plasmid (pcDNA3.1-TGF-β1-3×Flag, the plasmid concentration was 3-5 μg/mL) to enhance the secretion of TGF-β1 protein factor after cell transplantation, thereby increasing the survival rate and immunoregulatory effect of the cells.

A PD cell model was constructed using preformed fibrillar body (PFF) activated α-synuclein, microglia (BV2), and neurons (SH-SY5Y). The TGF-β1-overexpressing OM-MSCs were co-cultured with the PD cell model for 24 h, and the apoptosis of neurons, the dynamic immunoregulatory effect of microglia, and the scavenging capacity of α-synuclein in the PD cell model were detected by immunofluorescence and flow cytometry.

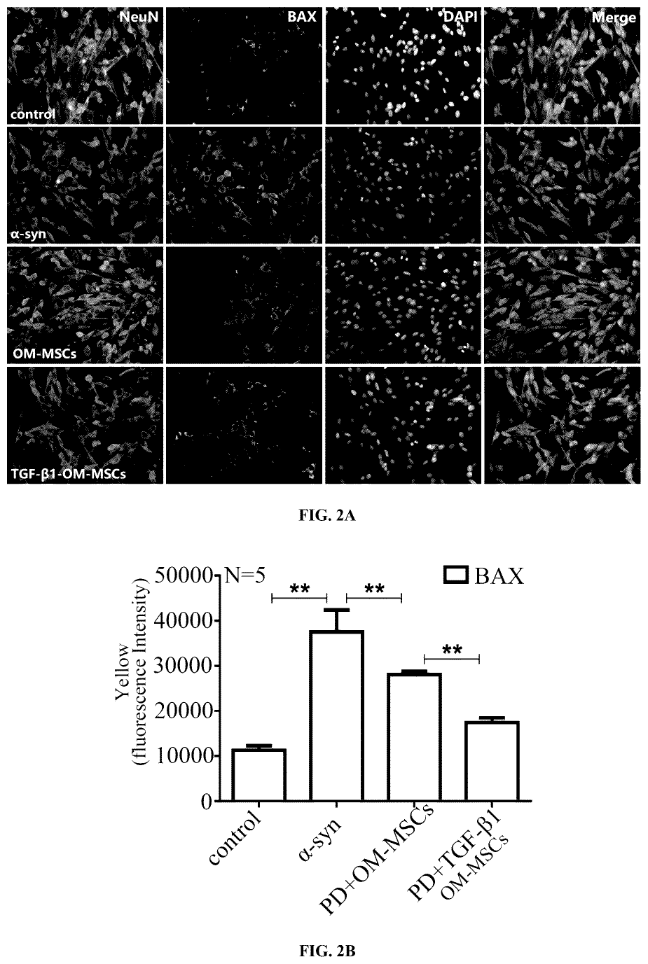

Specifically, in order to study the protective effect of OM-MSCs on neurons in the PD model, BV2 cells were placed in the upper chamber of a Transwell six-well plate device, while SH-SY5Y cells were placed in the lower chamber of the Transwell six-well plate device. Activated α-synuclein (250 nM) was added to a supernatant of the upper chamber and cultured for 1 h. After washed with medium 3 times, OM-MSCs (1×10⁵) before and after overexpression of TGF-β1 were added into the upper chamber containing BV2 cells to allow co-culture for 24 h. The experiment was divided into 4 groups: a control group (control), a model group (α-syn), an OM-MSCs intervention group (OM-MSCs), and a TGF-β1-overexpressing OM-MSCs intervention group (TGF-β1-OM-MSCs). The SH-SY5Y cells in each experimental group were double-stained with the neuron marker NeuN and the apoptosis marker BAX, and the yellow fluorescence intensity value after the fluorescence fusion of BAX and NeuN was statistically analyzed. The results showed that compared with those in the control group, the apoptosis of SH-SY5Y cells in the model group increased significantly, and the cytoskeleton morphology changed, resulting in axon shortening. The apoptosis of cells in the OM-MSCs intervention group was significantly reduced, and the cytoskeleton morphology was improved. Moreover, the apoptosis of cells in the TGF-β1-overexpressing OM-MSCs intervention group was reduced most significantly, the cytoskeleton morphology was also significantly improved, and axon elongation occurred (FIG. 2 shows the protective effect of OM-MSCs on DNs promoted by overexpression of TGF-β1, in which the left panel shows the fluorescence double staining of the neuron marker NeuN and the apoptosis marker BAX of SH-SY5Y cells in each group, while the right panel shows a bar plot of the left panel. The results suggested that compared with those in the control group, the apoptosis of SH-SY5Y cells in the model group increased significantly, and the cytoskeleton morphology changed, resulting in axon shortening. The apoptosis of cells in the OM-MSCs intervention group was significantly reduced, and the cytoskeleton morphology was improved. In addition, the apoptosis of cells in the TGF-β1-overexpressing OM-MSCs intervention group was reduced most significantly, the cytoskeleton morphology was also significantly improved, and axon elongation occurred. The above comparisons were all statistically significant (P<0.01)). This indicated that OM-MSCs had a certain protective effect on neurons, and the TGF-β1-overexpressing OM-MSCs showed a significantly-enhanced neuroprotective function.

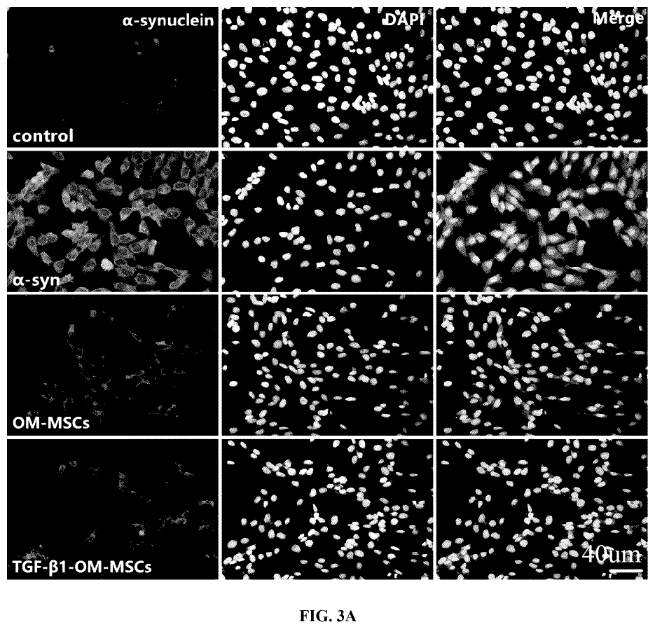

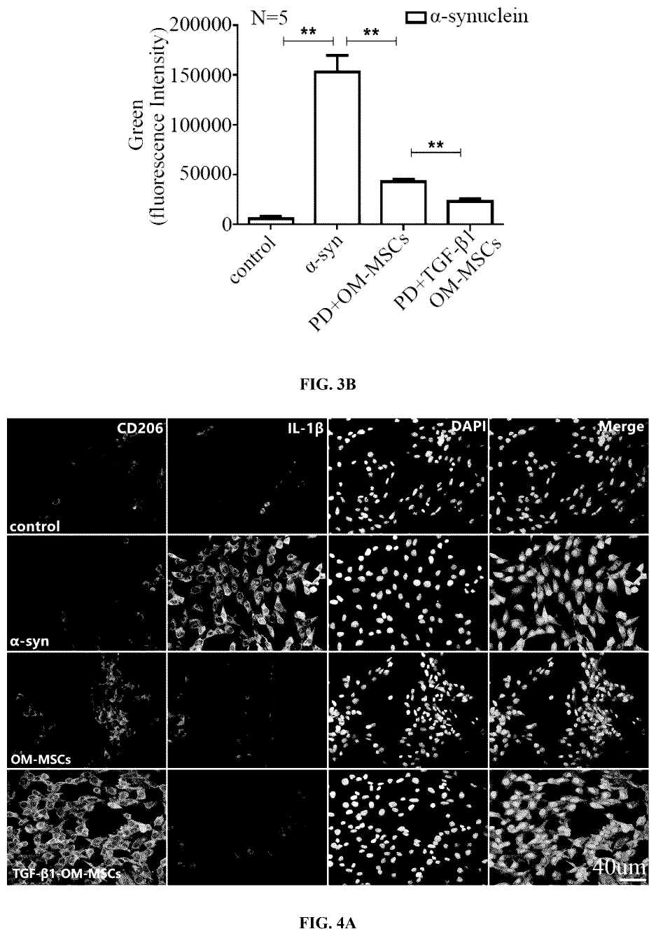

In order to investigate the capacity of OM-MSCs to scavenge α-synuclein from activated microglia, BV2 cells were placed in the upper chamber of a Transwell six-well plate device. Activated α-synuclein (250 nM) was added to a supernatant of the upper chamber and cultured for 1 h. After washing with medium 3 times, OM-MSCs (about 1×10⁵) before and after overexpression of TGF-β1 were added to the upper chamber of Transwell six-well plate and intervened by co-culture for 24 h. Experimental grouping was the same as above. The results of immunofluorescence showed that compared with that of the control group, the expression of α-synuclein in BV2 cells was upregulated in the model group, and there was a significant statistical difference (P<0.01). The OM-MSCs intervention group could reduce the expression of α-synuclein, while the scavenging capacity of the TGF-β1-overexpressing OM-MSCs intervention group was significantly improved (FIG. 3 shows the results of TGF-β1-overexpressing OM-MSCs to scavenge α-synuclein in microglia, where the left panel shows the α-syn fluorescence staining picture of BV2 cells, while the right panel shows a bar plot of the left panel. The results indicate that compared with that of the control group, the expression of α-syn in BV2 cells was upregulated, and there was a significant statistical difference (P<0.01). The OM-MSCs intervention group could reduce the expression of α-syn, and the scavenging capacity of the TGF-β1-overexpressing OM-MSCs intervention group was significantly improved, and there was a significant statistical difference (P<0.01)). This indicated that OM-MSCs had a certain scavenging capacity on α-synuclein in microglia, and the TGF-β1-overexpressing OM-MSCs had a significantly-enhanced scavenging capacity.

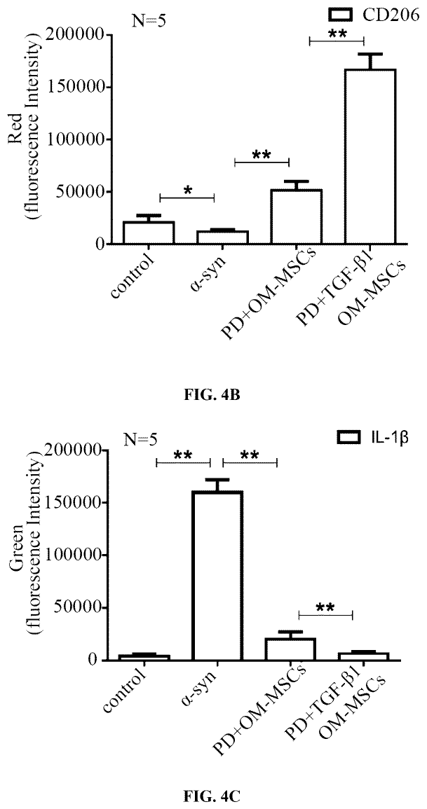

To further investigate the role of OM-MSCs in an immunoregulatory effect of the transformation of activated microglia from the pro-inflammatory M1 type to the anti-inflammatory M2 type, the anti-inflammatory phenotype marker CD206 and the pro-inflammatory phenotype marker IL-1β of BV2 cells in each group were detected by immunofluorescence. Experimental grouping and culture method were the same as above. Immunofluorescence results showed that compared with that of the control group, the expression of CD206 in BV2 cells was downregulated in the model group, with a statistically significant difference (P<0.05), while the expression of IL-1β was significantly upregulated, with a statistically significant difference (P<0.01). The OM-MSCs intervention group could significantly increase the expression of CD206 and significantly reduce the expression of IL-1β, and both had significant statistical differences (P<0.01), while the TGF-β1-overexpressing OM-MSCs intervention group had the most significant effect on upregulating CD206 and downregulating IL-1β expression (FIG. 4 shows the results of overexpression of TGF-β1 in promoting the immunoregulatory effect of OM-MSCs on microglia, where the upper panel shows the fluorescence double staining of IL-1β and CD206 in BV2 cells, and the lower panel shows the bar plots of the upper panel. The results indicate that compared with that of the control group, the expression of CD206 in BV2 cells in the model group was downregulated, with statistical differences (P<0.05), while the expression of IL-1β was significantly upregulated, with significant statistical differences (P<0.01). The OM-MSCs intervention group could significantly increase the expression of CD206 and significantly reduce the expression of IL-1β, and both had significant statistical differences (P<0.01), while the TGF-β1-overexpressing OM-MSCs intervention group had the most significant effects on upregulating CD206 and downregulating IL-1β, and both had significant statistical differences (P<0.01). This indicated that OM-MSCs could exert their immunoregulatory effect to promote the transformation of microglia from the pro-inflammatory M1 type to the anti-inflammatory M2 type, and the TGF-β1-overexpressing OM-MSCs had a more significant immunoregulatory effect.

In the present disclosure, a chronic PD mouse model was established by combining 1-methyl-4-phenyl-1,2,3,6-tetrahydropyridine (MPTP, 25 mg/kg) with probenecid (25 mg/kg). The TGF-β1-overexpressing OM-MSCs were transplanted into a lateral ventricle by stereotaxy, and the neurological function behavioral scores of the open field test and the rotarod test were conducted on the 7th day after transplantation; the loss of DNs in the substantia nigra of the PD mouse model was detected by immunohistochemistry.

To investigate the therapeutic effect of TGF-β1-overexpressing OM-MSCs on PD mouse model, the experiment was divided into: a sham operation group (sham), a model group (PD+phosphate-buffured saline (PBS)), an OM-MSCs treatment group (PD+OM-MSCs), and TGF-β1-overexpressing OM-MSCs treatment group (PD+TGF-β1-OM-MSCs). The transplantation time for each group was the second day after the last administration of the modeling, and the neurological function score and pathological examination were conducted on the seventh day after transplantation.

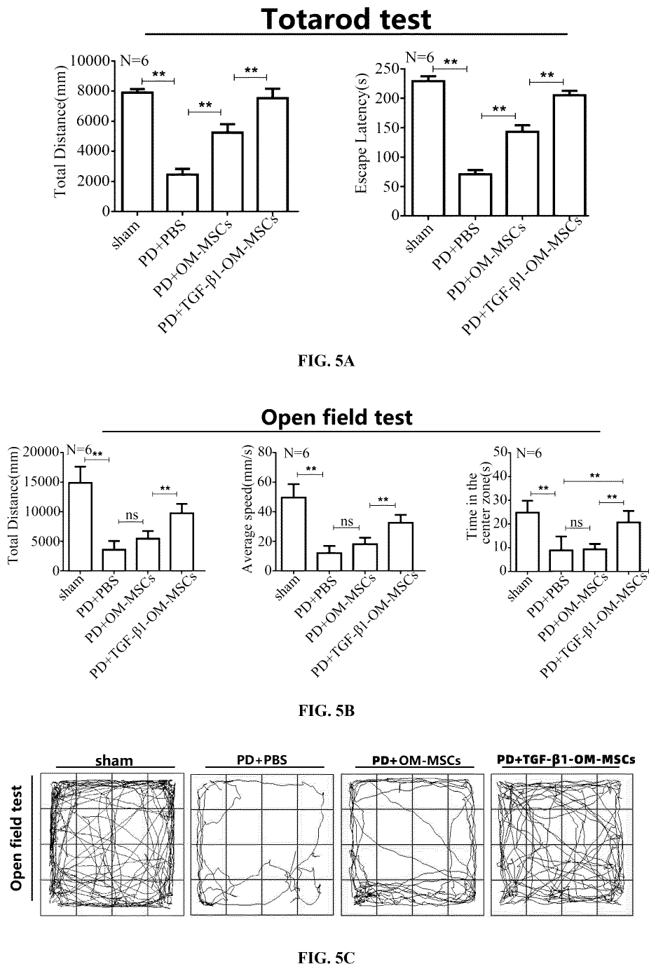

The neurological function scores of the experimental groups were conducted in the rotarod test and the open field test. The results of the passive motor neurological function score of the rotarod test showed that the latency and total distance of the model group were significantly reduced compared with those of the sham operation group, and significantly increased in the OM-MSCs treatment group, and the score of the TGF-β1-overexpressing OM-MSCs treatment group increased the most significantly (FIG. 5 shows the neurological function score results of the PD mouse model treated with TGF-β1-overexpressing OM-MSCs, in which the upper panel shows the rotarod test score statistics. The results indicate that the latency and total distance of the model group were significantly reduced compared with those of the sham operation group, and significantly increased in the OM-MSCs treatment group, while the score of the TGF-β1-overexpressing OM-MSCs treatment group increased most significantly. All differences were statistically significant (P<0.01). The middle panel shows the score statistical plots of open field test, and the lower panel shows trajectory charts of the open field test. The results indicate that the total distance, average speed, and central time of the model group were significantly reduced compared with those the sham operation group, and the OM-MSCs treatment group increased but there was no statistical difference, while the score of the TGF-β1-overexpressing OM-MSCs treatment group increased significantly, and the prevention group was the most significant, all of which were statistically significant (P<0.01). This indicated that OM-MSCs transplantation had a certain improvement effect on the neurological function of PD mouse models, and TGF-β1-overexpressing OM-MSCs intervention could significantly improve the neuromotor function of PD mouse models.

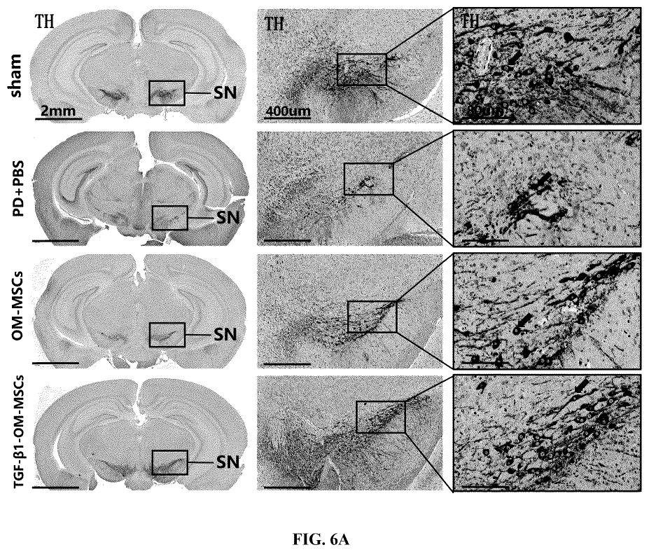

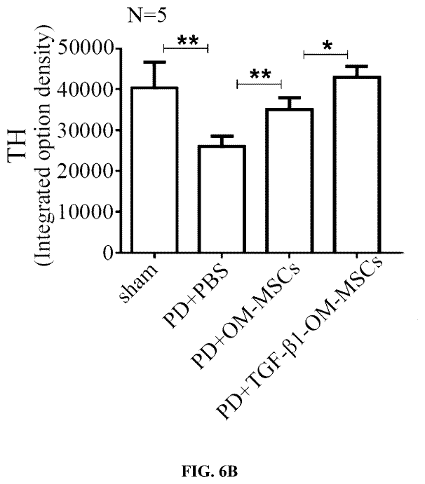

In order to explore the protective effect of OM-MSCs on DN function of PD mice, tyrosine hydroxylase (TH) immunohistochemistry was conducted on each experimental group, and then slide scanning was conducted to observe the loss of TH-positive cells in the substantia nigra. The results of immunohistochemistry showed that compared with that of the sham operation group, the number of TH-positive cells in the model group was significantly reduced, while that in the OM-MSCs treatment group was significantly increased, and that in the TGF-β1-overexpressing OM-MSCs group was most significantly increased (FIG. 6 shows the protective effect of TGF-β1-overexpressing OM-MSCs on DNs in the substantia nigra of PD mice, where the upper panel displays scanned imaged of slides after TH immunohistochemistry of each group, and the lower panel shows a bar plot of the upper panel. The results indicate that compared with that of the sham operation group, the number of TH-positive cells in the model group was significantly reduced, while that in the OM-MSCs treatment group was significantly increased, and that in the TGF-β1-overexpressing OM-MSCs group was the most significantly increased. All differences were statistically significant (P<0.01)). This indicated that TGF-β1-overexpressing OM-MSCs could significantly improve DN function in PD mouse model.

Although the present disclosure is described in detail in conjunction with the foregoing examples, they are only a part of, not all of, the embodiments of the present disclosure. Other embodiments can be obtained based on these examples without creative efforts, and all of these embodiments shall fall within the protection scope of the present disclosure.

1-4. (canceled)

5. A method of immunoregulation, comprising administering a transforming growth factor beta 1 (TGF-β1)-overexpressing olfactory mucosa mesenchymal stem cell (OM-MSC) to a subject in need thereof.

6. The method according to claim 5, wherein the immunoregulatory effect comprises promoting transformation of microglia from a pro-inflammatory M1 type into an anti-inflammatory M2 type.

7. The method according to claim 5, wherein a method for preparing the TGF-β1-overexpressing OM-MSC comprises: subjecting an OM-MSC to transfection with TGF-β1-overexpressing plasmid.

8. The method according to claim 7, wherein the TGF-β1-overexpressing plasmid is constructed based on a pcDNA3.1-3×Flag plasmid.

9. The method according to claim 7, wherein the transfection is conducted when the OM-MSC is passaged to a fourth generation.

10. The method according to claim 7, wherein the TGF-β1-overexpressing plasmid has a mass concentration of 3 μg/mL to 5 μg/mL and the OM-MSC has a viable count of 1×105 cells/10 μL during the transfection.

11. A method for treating PD, comprising: transplanting a TGF-β1-overexpressing OM-MSC into a lateral ventricle by stereotaxy.

12. A method for treating PD, comprising: administering a TGF-β1-overexpressing OM-MSC by intraspinal injection or intravenous injection.

13-14. (canceled)

15. The method according to claim 7, wherein the immunoregulatory effect comprises promoting transformation of microglia from a pro-inflammatory M1 type into an anti-inflammatory M2 type.
